(19) United States
(12) Patent Application Publication
(10) Pub. No.: US 2008/0163869 A1
(43) Pub. Date: Jul. 10, 2008

(54) Inhaling Apparatus And Liquid Agent Ejection Cartridge

(75) Inventors: Toshiyuki Nobutani, Yokohama-shi (JP); Mitsuru Imai, Chichibu-shi (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 11/814,083

(22) PCT Filed: Feb. 2, 2006
(86) PCT No.: PCT/JP2006/302214

§ 371 Date: Jul. 17, 2007

(30) Foreign Application Priority Data
Feb. 3, 2005 (JP) 2005-027912

(51) Int. Cl. A61M 11/00

(52) U.S. Cl. 128/200.23

(57) ABSTRACT

An inhaling apparatus is provided with a fitting section for removably fitting thereto a liquid agent ejection cartridge. The liquid agent ejection cartridge comprises storage means for storing a liquid agent, ejection means for ejecting the liquid agent as liquid droplets, a suction port section to be held in the mouth of a user inhaling the liquid agent, and a liquid agent flow path for connecting the ejection means and the suction port section. The apparatus is portable and the cartridge is disposable.

### TECHNICAL FIELD

This invention relates to an inhaling apparatus and a liquid agent ejection cartridge to be mounted in the same. More particularly, the present invention relates to an inhaling apparatus and a liquid agent ejection cartridge for ejecting a medicine, an aromatic, nicotine or some other liking of a user in very small droplets so as to make the user inhale it.

### BACKGROUND ART

Our society is aging as the average life span of people is extended due to the advancement of science including medical science. On the other hand, new infectious diseases and other diseases have been and being discovered mainly because of the changes in diet and in the living environment, environmental pollutions, novel viruses and microbes and other factors to make people uneasy about their health. Particularly, the number of patients suffering from life-style related diseases such as diabetes and hypertension is increasing to make a social issue in so-called advanced countries.

For example, insulin has to be administered to diabetic patients. Conventionally, insulin is administered to the patient mostly by means of injection at every meal. However, administration of medicines by injection forces a considerable pain on the part of patients. As a solution of this problem, administration of medicines by way of the respiratory system has been envisaged. Three techniques are available for it. They include the use of a metered dose inhaler, that of a dry powder inhaler and that of an atomizer.

Metered dose inhalers (MDIs) are being popularly used for asthmatic patients. An MDI is equipped with a valve for ejecting a metered dose of aerosol in operation. The main body of the MDI can be made very compact to make it convenient and portable. However, the dose by ejection can vary considerably. Additionally, the use of an MDI requires a certain extent of synchronization of the manual operation of the valve and the inhalation but many users feel it difficult to synchronize them.

With a dry powder inhaler (DPI), the patient is required to inhale a large amount of air to fluidize a powdery medicine and administer it to the inside of the bronchial system. While it may seem that the DPI can get rid of the problem of synchronization of the valve operation and the inhalation of the medicine of the MDI, it is a severe burden on the part of patients to inhale a large amount of air with the DPI. Additionally, the DPI can trigger a fit of asthma if the patient is sensitive to moisture and also to the inhaled powder. Thus, a DPI cannot be used for such a patient. Additionally, the inhaling power varies from person to person and hence the dosage can vary from person to person.

An atomizer is adapted to generate an aerosol by atomizing liquid by means of a carrier gas flow. It requires a gas compressor that operates continuously and a large amount of compressed gas. Generally, the size of droplets of aerosol is a function of the pressure and the velocity of the carrier gas and hence it is not easy to independently change the density of the medicine in the gas flow. Additionally, inhalation reduces the pressure in the inside of the nozzle of the atomizer and hence the dose and the particle size are influenced by the span and the intensity of each breathing action.

As pointed out above, all the described known apparatus are accompanied by the problem of accuracy of administering a right quantity of medicine to the right position with a right particle size. In other words, they can find applications only for medicines having a large tolerance in terms of dosage. Additionally, they resort to the user's skill when a medicine has to be administered to the right target position in the patient's body.

Meanwhile, there is a demand for improved administration systems that can optimize the effect of medical treatment in the nose and the lung by means of a locally effective medicine. Additionally, as a result of the advancement of medical science in recent years, it has been found that administration of medicines such as proteins, peptides, anodynes and other agents to the lung (pulmonary administration) is considerably advantageous if compared with conventional oral administration or administration by injection. However, known inhalers show a large dispersion in terms of particle size and dosage and hence they are not suited for the above listed applications.

This problem will be described further by way of a specific example. Of the diabetic patients, the number of which is currently increasing, those of the so-called type I who are dependent on insulin need to be periodically administered with insulin because they do not secrete insulin from the pancreas. Insulin is currently administered by subcutaneous injection to put a large physical and mental burden on the patient. Pen-type injection syringes that come with a fine needle and do not give a large pain to the patient have been developed in order to alleviate the burden of the patient. However, many type I diabetic patents are working with ordinary healthy people except that they need to be administered with insulin periodically. In other words, it will be mentally cruel to such a patient to be administered with insulin by injection in front of people and hence it will be difficult for such a patient to be administered at right time.

Thus, there is a strong demand for a technique with which the patient can administer a medicine by him- or herself with ease in such a way that the medicine is ejected not by injection but as liquid droplets so that it can get to the lung with a breathing and the medicine is taken in by pulmonary administration.

Techniques for ejecting a predetermined number of appropriately sized droplets of a physiologically effective medicine through an ejection orifice into an air flow that is to be inhaled by way of a mouthpiece or the like under the effect of a Bubblejet (tradename) or a piezoelectric element arranged in an ejection head (ejector) have been proposed (see, inter alia, International Patent Publication No. WO95/01137 and International Patent Publication No. WO02/04043). Japanese Patent Application Laid-Open Publication No. 2003-154655 proposes a liquid ejection head for ejecting micro-droplets of the size of the order of sub-picoliter that are suited for pulmonary inhalation.

### DISCLOSURE OF THE INVENTION

However, in any of the known inhaling apparatus, while the ejection head section and the liquid agent storage section are integrally formed (such an integral combination is referred to as head cartridge hereinafter), the mouthpiece is a separate unit that is designed to be fitted to the ejection head section. With such an arrangement, once the mouthpiece is used for inhalation, the drawn out liquid agent partly adheres to the inside of the mouthpiece and also to the flow path (which is arranged in the inhaling apparatus main body) linked to the mouthpiece to give rise to a situation that is not favorable from the sanitary viewpoint. If the mouthpiece and the internal flow path have to be washed before they are used again, it means that they cannot be used wherever they are needed. Thus, the known inhaling apparatus are considerably cumbersome to users. Particularly, mold and the like can come out when a known inhaling apparatus is used for inhaling a protein-based agent so that the head cartridge has to be discarded like syringe needles. Therefore, the mouthpiece has to be discarded or, if it is to be reused, it has to be inevitably washed to put a considerable burden on the part of the user. Then, the user of such an inhaling apparatus is compelled to pay attention to sanitation. In other words, known inhaling apparatus cannot be used in a simple and easy manner.

In view of the above-identified circumstances, the present invention provides an inhaling apparatus provided with a fitting section for removably fitting thereto a liquid agent ejection cartridge as an integral unit, the liquid agent ejection cartridge comprising:

storage means for storing a liquid agent;

ejection means for ejecting the liquid agent as liquid droplets;

a suction port section; and

a liquid agent flow path connecting the ejection means and the suction port section,

said apparatus being adapted for a user to inhale the liquid agent from it by way of the suction port section.

In another aspect of the present invention, there is provided a liquid agent ejection cartridge comprising as integral parts:

storage means for storing a liquid agent;

ejection means for ejecting the liquid agent as liquid droplets;

a suction port section to be held in the mouth of a user inhaling the liquid agent; and

a liquid agent flow path for connecting the ejection means and the suction port section.

The liquid agent ejection cartridge may be formed of a head cartridge including the storage means and the ejection means and a mouthpiece including the flow path and the suction port section so long as it is integrally fitted to the inhaling apparatus.

Thus, according to the invention, the head cartridge and the mouthpiece are put together to form an integral unit so that they can be easily and removably fitted to an inhaling apparatus. With such an arrangement, the head cartridge and the mouthpiece can be removed from the inhaling apparatus as a unit and can be discarded as the unit after use to alleviate the burden on the part of the user. Thus, the present invention provides an inhaling apparatus that the user can use with ease.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a schematic cross sectional view of an embodiment of mouthpiece-incorporating integral type head cartridge according to the invention;

FIG. 2 is a schematic perspective view of the embodiment of mouthpiece-incorporating integral type head cartridge of FIG. 1;

FIG. 3 is a schematic perspective view of the main body of an embodiment of inhaling apparatus according to the invention;

FIG. 4 is a schematic cross sectional view of the main body of the embodiment of inhaling apparatus of FIG. 3 to which a mouthpiece-incorporating integral type head cartridge is fitted;

FIG. 5 is a graph schematically illustrating the relationship between inhalation by the user and ejection of a liquid agent;

FIG. 6 is a schematic block diagram of the control circuit of an embodiment of inhaling apparatus according to the invention; and

FIG. 7 is a schematic perspective view of another embodiment of inhaling apparatus where the mouthpiece and the head cartridge are separable.

### BEST MODES FOR CARRYING OUT THE INVENTION

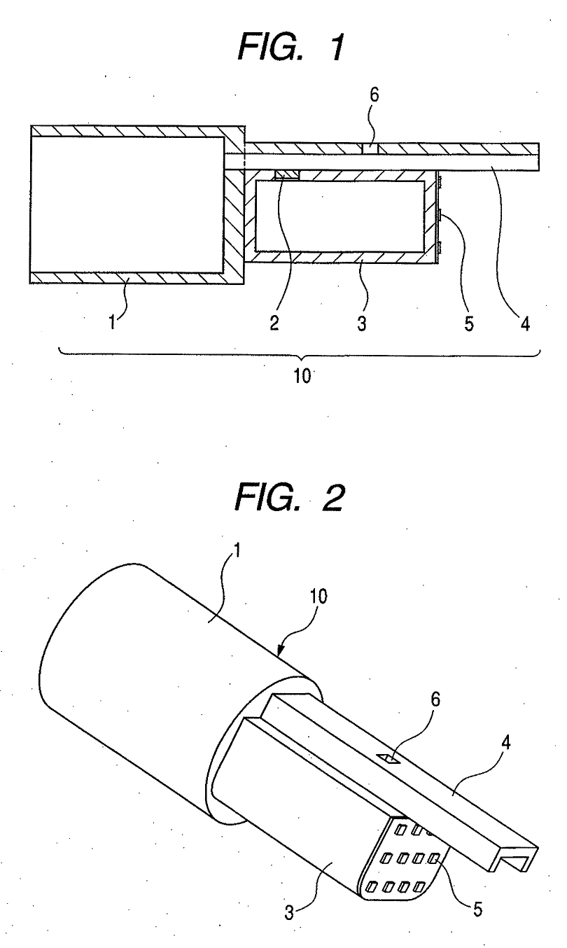

Now, preferred embodiments of this invention will be described in detail by referring to FIGS. 1 through 7.

FIG. 1 is a schematic cross sectional view of an embodiment of mouthpiece-incorporating integral type head cartridge (liquid ejection cartridge) according to the invention and FIG. 2 is a schematic perspective view of the embodiment of mouthpiece-incorporating integral type head cartridge, showing the profile thereof. Referring to FIGS. 1 and 2, the mouthpiece-incorporating integral type head cartridge 10 comprises a mouthpiece suction port section 1 and an ejection head (ejection means) 2. The ejection head 2 has one or more than one orifices for ejecting liquid droplets by means of a thermal ink-jet system. An ink-jet head of the type comprising a heater or a piezoelectric element or a liquid ejection head having a mesh structure with a large number of pores that is adapted to be used in the field of inhaling apparatus may be used for the ejection head 2.

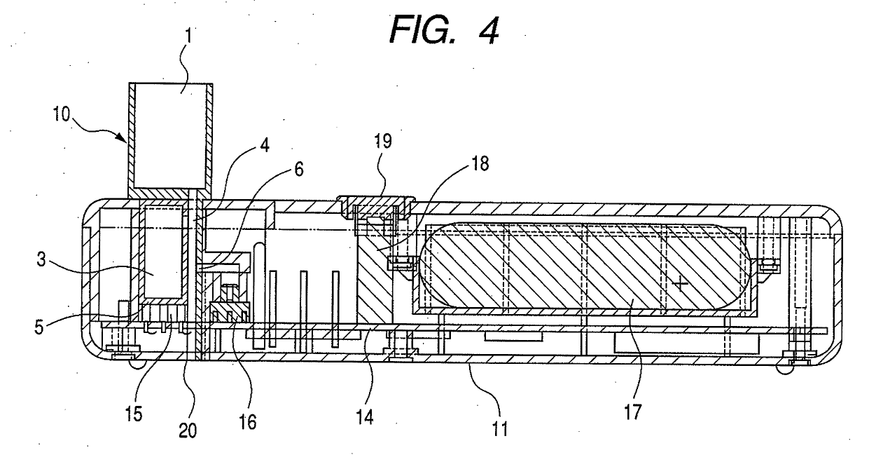

Referring to FIGS. 1 and 2, the mouthpiece-incorporating integral type head cartridge 10 further comprises a liquid agent storage tank (liquid agent storage means) 3 and a flow path 4 for air and liquid droplets (of the liquid agent), which flow path 4 shows a U-shaped cross section. As a whole, the flow path 4 is formed at the outer surface of the suction part section 1 of the mouthpiece and that of the liquid agent storage tank 3 located at the side of the ejection head 2 and connected to the air intake port 20 arranged at the rear side of the inhaling apparatus main body 11 illustrated in FIG. 4 to take in external air. In FIGS. 1 and 2, numeral 5 denotes an electric connector member, which is a member having an electric connection surface for supplying electric power to the heater arranged in the ejection head 2 so as to generate thermal energy.

A hole 6 that communicates with the negative pressure gauging hole of a negative pressure sensor 16 is arranged midway on the flow path 4. As shown in FIG. 4, the negative pressure sensor 16 arranged on a control substrate 14 is designed to gauge the flow rate of air flowing through the flow path 4 extending from the air intake port 20 to the mouthpiece suction port section 1.

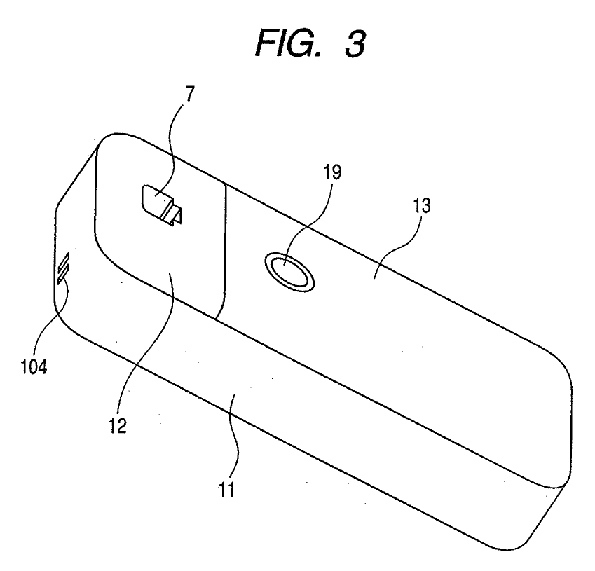

FIG. 3 is a schematic perspective view of the inhaling apparatus main body, showing the state thereof before a mouthpiece-incorporating integral type head cartridge 10 is fitted to it. Referring to FIG. 3, there are shown an inhaling apparatus main body 11, a head cartridge guide 12 and a front cover 13. The housing of the inhaling apparatus is formed by these components. The head cartridge guide 12 is provided with a liquid agent ejection cartridge fitting port 7. A mouthpiece-incorporating integral type head cartridge 10 is inserted and fitted from the side of the flow path 4. The internal profile of the liquid agent ejection cartridge fitting port 7 is so designed that, when the mouthpiece-incorporating integral type head cartridge 10 is inserted into the liquid agent ejection cartridge fitting port 7 from the side of the flow path 4, the flow path 4 is connected to the air intake port 20 and the gauging hole of the negative pressure sensor 16 and the hole 6 are held in communication with each other so that the mouthpiece suction port section 1 is reliably put into a predetermined state where it is projecting to the outside as shown in FIG. 4. In short, the mouthpiece-incorporating integral type head cartridge shows a profile that fits in the fitting section of the inhaling apparatus.

FIG. 4 is schematic cross sectional view of the inhaling apparatus main body to which a mouthpiece-incorporating integral type head cartridge 10 is fitted. A control substrate 14 for controlling the inhaling apparatus main body 11 is arranged under the battery 17 for supplying electric power to the ejection head 2. The control substrate 14 is provided with a contact probe 15 for supplying heat-generating electric power to the ejection head 2 of the head cartridge 10. When a mouthpiece-incorporating integral type head cartridge 10 is fitted to the liquid agent ejection cartridge fitting port 7, the contact probe 15 is reliably connected to the electric connection member 5 in a predetermined mode.

As the user who holds the suction port section 1 of the inhaling apparatus that is in the state as illustrated in FIG. 4 takes an inhaling action, air is taken into the flow path 4 from the air intake port 20 to form a mixture fluid with the liquid agent, or the medicinal agent, ejected from the ejection port arranged at the ejection head 2 of the head cartridge 10, which mixture fluid is then directed toward the suction port section 1 of the mouthpiece that is profiled so as to be snugly held in the mouth of the user. The suction port section 1 of the mouthpiece is made to show an elliptic profile that matches the profile of the mouth of man. In short, it shows a profile that matches the profile of the mouth of a man so that the mixture fluid may be prevented from leaking out through the side parts of the mouth to waste the mixture fluid and the liquid agent may be efficiently taken into the body.

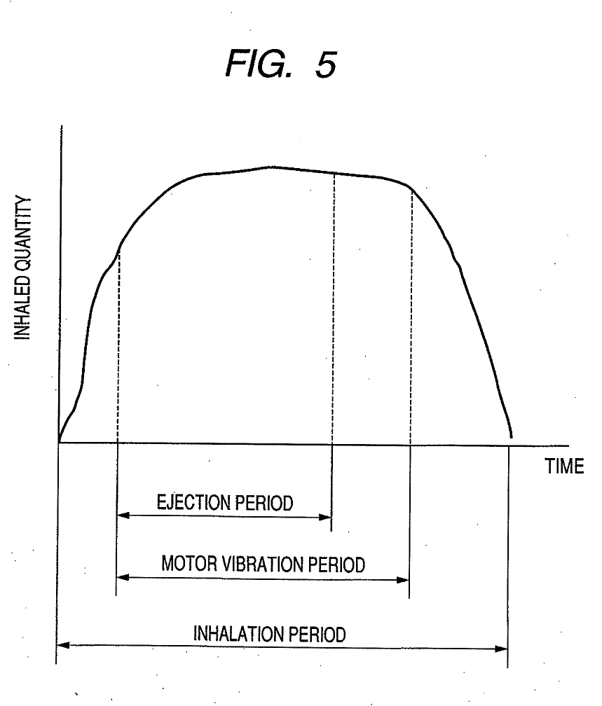

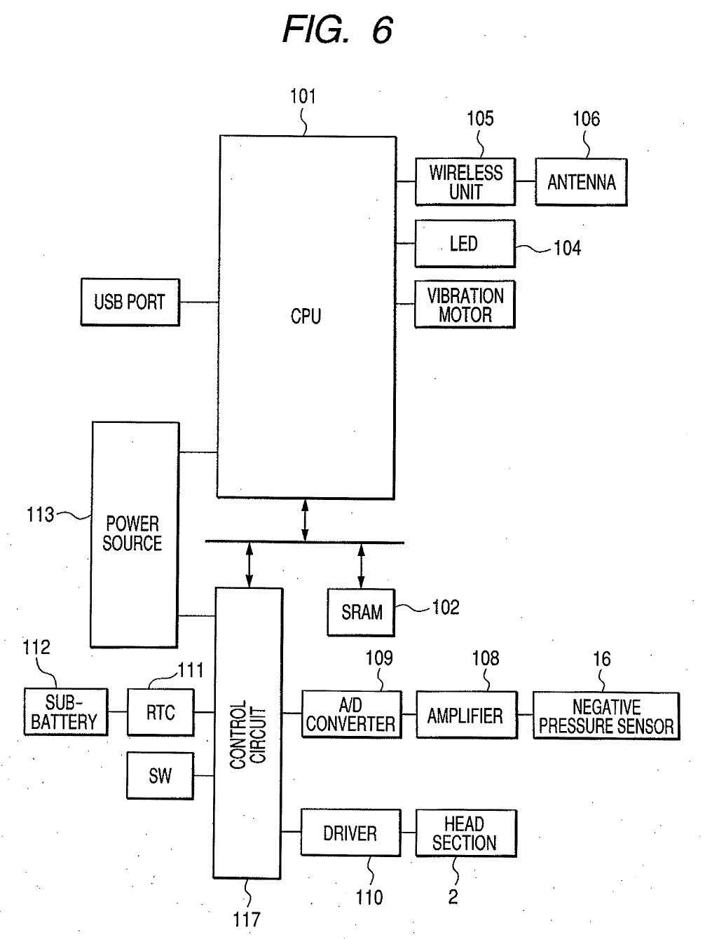

A vibration motor (not shown) is arranged on the control substrate 14. The control substrate 14 controls the operation of the inhaling apparatus in such a way that, when an inhaling action is started and the flow rate gets to a level that allows the liquid agent to be ejected, the ejection head 2 starts ejecting the liquid agent and, at the same time, the vibration motor start vibrating in order to notify the user of the start of ejection. The vibration motor keeps on vibrating for a predetermined spare inhalation period so that, after a predetermined quantity of the liquid agent is ejected, a spare quantity of the liquid agent that is computationally determined on the basis of the inhaling rate computed from the reading of the negative pressure sensor 16 and other factors may be inhaled by the user and the last drop of the ejected liquid agent may gets to the lung of the user. In this way, the user is encouraged to inhale the liquid agent until the liquid agent, or the medicinal agent, completely gets to the lung. The user stops his or her inhaling action when the vibration motor stops vibrating. FIG. 5 is a graph showing an inhalation curve that illustrates the relationship between the inhaled quantity of a liquid agent and the ejection period, the motor vibration period and the inhalation period.

FIG. 6 is a schematic block diagram of the control substrate 14 of the embodiment and its periphery. Referring to FIG. 6, the control substrate 14 and its periphery carry a CPU 101 that is a processor controller containing a flash ROM for storing a program, an SRAM 102 that is a readable/writable memory for temporarily storing data when the program is executed, an LED 104 (see FIG. 3) that is a display unit for notifying a user, a maintenance engineer or the like of the status of the apparatus, a wireless unit 105 for transmitting data on the status of the apparatus and the data stored in the memory to a host and receiving data from the host, an antenna 106 to be used for the wireless unit, an amplifier 108 for changing and amplifying the output level of the negative pressure sensor 16, an A/D converter 109 for converting the analog output of the amplifier 108 into digital signals, a driver 110 for controlling the head section 2, an RTC (real time clock) 111 that operates as calendar and clock, a backup battery 112 for the RTC 111, a power source 113 for producing various voltages to be supplied to the electric circuits of the apparatus that includes a main battery, a charging circuit, a reset circuit and a power supply switch 18, and a control circuit 117 for processing output signals to and input signal from various blocks that is connected to the CPU 101 by way of a bus. A USB port that is connected to the CPU 101 is also provided as interface.

With the above-described arrangement, as the power supply switch 18 is operated by way of a power supply button 19, the power source 113 outputs a reset signal to the CPU 101 and the CPU 101 is initialized by the signal. Then, the program stored in the internal flash ROM starts operating in an ordinary operation mode unless some other mode of operation is specified. As the user inhales, the output of the negative pressure sensor 16 changes and the change is transmitted to the CPU 101 by way of the amplifier 108, the A/D converter 109 and the control circuit 117. When the inhaled quantity exceeds a predetermined threshold value, the CPU 101 applies a voltage to the vibration motor in the inhaling apparatus main body to make it vibrate and, at the same time, transmits a pulse signal to the ejection head (head section) 2 by way of the driver 110 so that the liquid agent contained in the liquid agent ejection cartridge 10 is ejected. After the liquid agent is ejected for a preset period of time, the vibration motor keeps on vibrating for a preset vibration period in order to encourage the user to keep on inhaling (see, FIG. 5).

With the above-described embodiment, a mouthpiece-incorporating integral type head cartridge can be freely and removably fitted to the inhaling apparatus. Then, the mouthpiece-incorporating integral type head cartridge can be disposed as waste to alleviate the mental and physical burden of the user when the inhaling process is over. Additionally, the inhaling apparatus is portable so that the user can conveniently carry it with him or her to anywhere and use it with ease anytime he or she needs to use. Since the air intake port 20 of the inhaling apparatus main body is free from contamination because only air passes through it so that the main body does not require washing. Thus, the head cartridge, the mouthpiece and the inhaling apparatus main body do not require any washing operation. In other words, the present invention provides a clean inhaling apparatus that the user can carry to anywhere and use with ease.

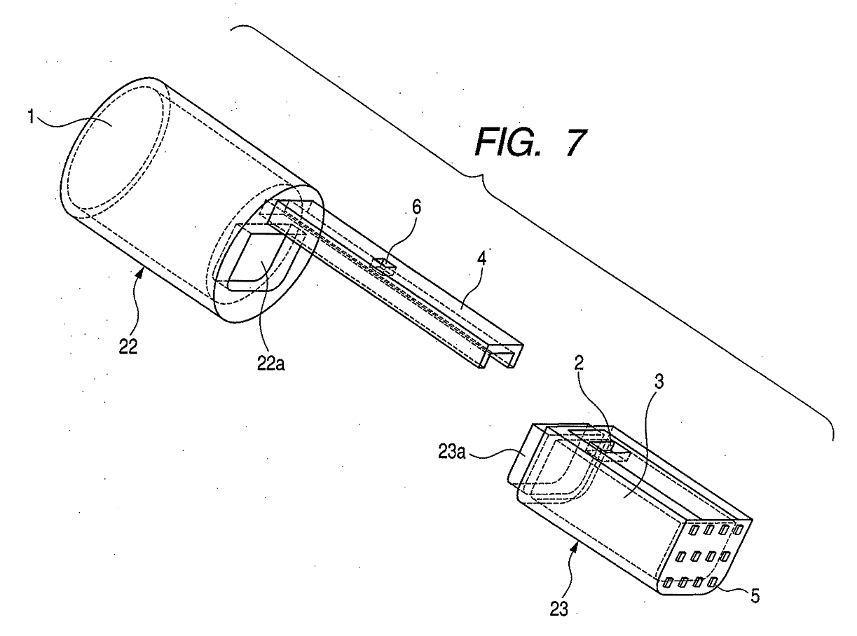

FIG. 7 is a schematic perspective view of another embodiment of mouthpiece-incorporating integral type head cartridge (liquid ejection cartridge) that is not integral before use but can be fitted to an inhaling apparatus main body as an integral unit. In FIG. 7, the mouthpiece 22 and the head cartridge 23 are separated yet. The mouthpiece 22 has a recessed section 22a while the head cartridge 23 has a projecting section 23a. When the embodiment is put to use, the recessed section 22a of the mouthpiece 22 and the projecting section 23a of the head cartridge 23 are brought into engagement with each other so that the mouthpiece 22 and the head cartridge 23 are put together to form an integral unit to be fitted to an inhaling apparatus main body. The integral unit can be taken out from the inhaling apparatus main body as it is so that the net effect thereof will be the same as that of the above described embodiment. The mouthpiece 22 may be molded by using a resin material.

Thus, a mouthpiece-incorporating integral type head cartridge according to the invention has a fitting section that allows the head cartridge to be fitted to an inhaling apparatus main body as an integral unit so that the burden on the part of the user is greatly reduced if compared with an arrangement using members having respective fitting sections because the user is not required to make efforts for fitting the members separately and correctly to an inhaling apparatus main body without confusion and mis-installing.

Since the mouthpiece and the flow path are designed to show dimensions that are suitable for inhaling actions. Thus, if the head section, the ejecting section and other sections are downsized further, the user can easily pick up the flow path section or the mouthpiece section of the integral type head cartridge with fingers and hence will not face any difficulty when putting the head cartridge in position.

Additionally, since the head cartridge is an integral type, the problem that the user can inadvertently touch the head section (the ejection port side in particular) and/or the electric contact points to adversely affect the ejection of the liquid agent is remarkably reduced.

Still additionally, since a mouthpiece-incorporating integral type head cartridge according to the invention can be made disposable, the mouthpiece 22 thereof may be produced by using paper and carried by the user in a carrying case or the like in a folded state so that the user may unfold it and combine it with a head cartridge 23 when he or she uses it. Such an arrangement is realized at low cost and made conveniently portable.

The present invention is not limited to the above embodiments and various changes and modifications can be made within the spirit and scope of the present invention. Therefore, to apprise the public of the scope of the present invention, the following claims are made.

This application claims priority from Japanese Patent Application No. 2005-027912 filed on Feb. 3, 2005, which is hereby incorporated by reference herein.

1-7. (canceled)

8. A liquid agent ejection cartridge comprising as integral parts:
storage means for storing a liquid agent;
ejection means for ejecting the liquid agent as liquid droplets;
a suction port section to be held in the mouth of a user inhaling the liquid agent; and
a liquid agent flow path connecting said ejection means and said suction port section,
wherein said storage means is located outside said liquid agent flow path.

9. The cartridge according to claim 8, wherein a head cartridge including said storage means and said ejection means and a mouthpiece including said liquid agent flow path and said suction port section are removably fitted to each other.

10. The cartridge according to claim 9, wherein the mouthpiece is made of paper and foldable.

11. The cartridge according to claim 8, wherein said ejection means ejects a liquid agent by means of an inkjet system.

12. An inhaling apparatus comprising a liquid agent ejection cartridge according to claim 8 and a fitting section for removably fitting thereto said liquid agent ejection cartridge as an integral unit.

13. The apparatus according to claim 12, wherein said apparatus is further provided with a control circuit for controlling ejection of said ejection means.

14. The apparatus according to claim 12, wherein said apparatus has an aperture section for fitting said cartridge to said fitting section.
